(19) United States
(12) Patent Application Publication
(10) Pub. No.: US 2005/0064479 A1
(43) Pub. Date: Mar. 24, 2005

(54) Compounds and methods for post incorporation labeling of nucleic acids

(75) Inventors: Glenn H. McGall, Palo Alto, CA (US); Anthony D. Barone, San Jose, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/916,849

(22) Filed: Aug. 12, 2004

Related U.S. Application Data
(60) Provisional application No. 60/494,874, filed on Aug. 12, 2003.

(51) Int. Cl. C12Q 1/68; C07H 21/04; C12P 19/34

(52) U.S. Cl. 435/6; 435/91.2; 536/25.3

(57) ABSTRACT

Methods are provided for post incorporation labeling of a nucleic acid, including for example cRNA, labeled with nucleotide analogs having a formula selected from the group consisting of 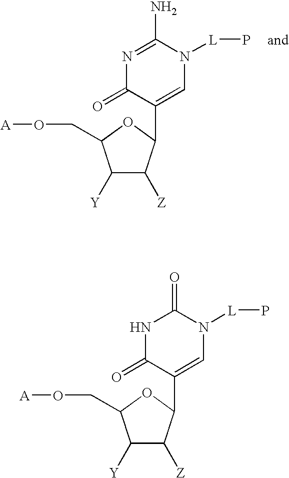 wherein A is H or a functional group that permits the attachment of the nucleic acid labeling compound to a nucleic acid; Y and Z are independently H or OH; L is linker group; and P is a connecting group. After incorporation of the nucleic acid (including cRNA) with the above nucleotide analogs, the nucleic acid is labeled with a detectable group reagent wherein said detetable group reagent comprises a chemical moiety which is capable of specifically reacting with said P group to allow coupling of the detectable group to said primed cRNA. Compounds comprising nucleotide analogs are also presented in accordance with the present invention. Methods are also presented for incorporating these compounds into nucleic acids and subsequently labeling the incorporated nucleotide analog with detectable moiety reagent.

### PRIORITY CLAIM

This Application claims the priority of U.S. Provisional Application Ser. No. 60/494,874 filed on Aug. 12, 2003. This application is incorporated herein by reference in its entirety for all purposes.

### BACKGROUND OF THE INVENTION

High density nucleic acid arrays (also called microarrays) have become widely used tools for monitoring gene expression both qualitatively and quantitatively to analyze patterns of RNA expression. Through microarrays, one can simultaneously analyze the expression of the entire set of genes involved with a biochemical pathway or disease state (e.g., cancer). Microarrays have also be widely employed genotyping and re-sequencing such as for example testing whether a donor had a particular SNP or has detectable levels of HIV.

Microarrays may be constructed by a variety of techniques and methodologies: At one end of the spectrum, cDNAs may be attached to a solid support and probed for hybridization with oligonucleotides or other nucleic acids. Higher density arrays may be fabricated of oligonucleotides of bound by a variety of techniques to a solid support or other medium. One particularly preferred and successful format of nucleic acid arrays is the GeneChip® Array of Affymetrix, Inc. of Santa Clara, Calif., which provides high density oligonucleotide arrays fabricated using photolithography.

For any nucleic acid array to be useful, there must be a way to determine whether complementary nucleic acid is bound to a particular probe. This is typically done by placing some type of label (e.g., a fluorescent tag), which allows areas of hybridization to be visualized by exposing a tag to a first wave length of light, followed by monitoring for fluorescent light having a longer wave length of light from the exposed tags.

Before the mid-1980's, nucleic acids were typically labeled isotopically. This technology, however, has largely by replaced by fluorescent labeling of nucleic acid. Fluor-labels may be specifically incorporated into nucleic acids either chemically or enzymatically. Moreover, it has been found that fluorescent labeled nucleotides substantially retain their ability to act as specific substrates of enzymes involved in a wide spectrum of nucleic acid synthesis, repair, transcription, replication, etc., allowing specific incorporation into a nucleic acid of fluorescently labeled nucleotides.

The present invention is directed to methods and compounds for nucleic acid labeling for, inter alia, use in nucleic acid and microarray analysis.

### SUMMARY OF THE INVENTION

Methods are presented for post-incorporation labeling of cRNA, the method having the steps of providing a cDNA template having a T7 RNA promoter, transcribing the template with a mixture of nucleotides, the mixture comprising a nucleotide analog, the analog selected from the group consisting of 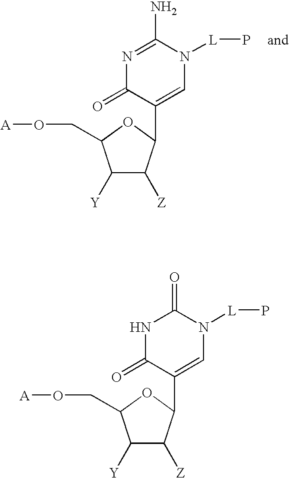
wherein A is triphosphate or α-thio triphosphate that permits the attachment of the nucleotide analog to a nucleic acid; Y and Z are independently H or OH; L is linker group; and P is a connecting group to provide primed cRNA; and reacting the primed cRNA with a detectable group reagent, wherein said detetable group comprises a chemical moiety which is capable of specifically reacting with said P group to allow coupling of the detectable group to said primed cRNA.

In preferred embodiments of the present invention the P group comprises a terminal moiety selected from the group consisting of —NH₂, —SH, —ONH₂, —CO₂H, —C(O)R, wherein R is H, alkyl, aryl or functionalized alkyl group and —C(R)HX, wherein X is a halogen. In accordance with one aspect of the present invention, the chemical moiety of the detectable group may chosen from the same group as set forth above for P.

In preferred embodiments of the disclosed invention present invention, L-P is

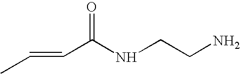

In the preferred method, it is also preferred that the nucleotides are

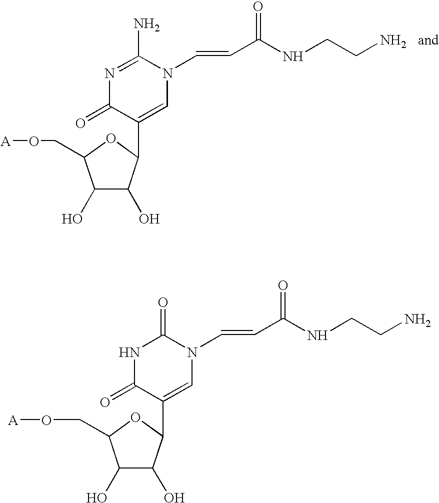

Compounds are also presented in accordance with the present invention, the compounds comprising nucleotide analogs have the following structures: 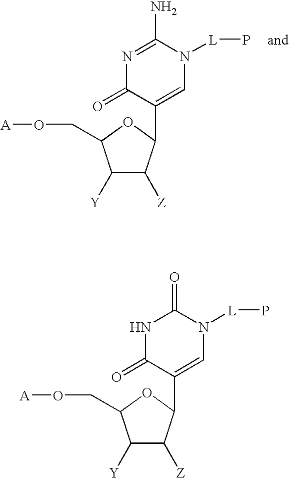
wherein A is H or a functional group that permits the attachment of the nucleotide analog to a nucleic acid; Y and Z are independently H or OH; L is linker group; and P is a connecting group.

With respect to the nucleotide analogs it is preferred that -L-P is

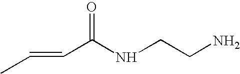

The compounds set forth below are particularly preferred.

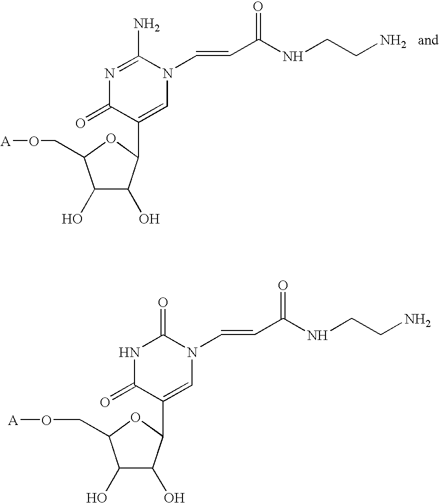

The instant invention also discloses various other methods and compounds as disclosed more fully below.

### DESCRIPTION OF THE PREFERRED EMBODIMENT

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited. As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer (anyone have the cite), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US 01/04285, and in U.S. patent applications Ser. No. 09/501,099 and Ser. No. 09/122,216 which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefor are shown in U.S. Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. For example, see the patents in the gene expression, profiling, genotyping and other use patents above, as well as U.S. Ser. No. 09/854,317, Wu and Wallace, Genomics 4, 560 (1989), Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), Guatelli et al., Proc. Nat. Acad. Sci. USA, 87, 1874 (1990), WO 88/10315, WO 90/06995, and U.S. Pat. No. 6,361,947.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625 and in PCT Application PCT/US99/06097 (published as WO99/47964), each of which also is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over the internet. See provisional application 60/349,546.

All patents, patent applications and publications cited in this application are hereby incorporated by reference in their entirety for all purposes to the same extent as if each individual patent, patent application or publication were so individually denoted.

Definitions

“Alkyl” refers to a straight chain, branched or cyclic chemical group containing only carbon and hydrogen. Alkyl groups include, without limitation, ethyl, propyl, butyl, pentyl, cyclopentyl and 2-methylbutyl. Alkyl groups are unsubstituted or substituted with 1 or more substituents (e.g., halogen, alkoxy, amino).

“Aryl” refers to a monovalent, unsaturated aromatic carbocyclic group. Aryl groups include, without limitation, phenyl, naphthyl, anthryl and biphenyl. Aryl groups are unsubstituted or substituted with 1 or more substituents (e.g. halogen, alkoxy, amino).

“Amido alkyl” refers to a chemical group having the structure —C(O)NR₃R₄—, wherein R₃ is hydrogen, alkyl or aryl, and R₄ is alkyl or aryl. Preferably, the amido alkyl group is of the structure —C(O)NH(CH₂)ₙR₅—, wherein n is an integer ranging from about 2 to about 10, and R₅ is O, NR₆, or C(O), and wherein R₆ is hydrogen, alkyl or aryl. More preferably, the amido alkyl group is of the structure —C(O)NH(CH₂)ₙN(H)—, wherein n is an integer ranging from about 2 to about 6. Most preferably, the amido alkyl group is of the structure —C(O)NH(CH₂)₄N(H)—.

“Alkynyl alkyl” refers to a chemical group having the structure —C≡C—R₄—, wherein R₄ is alkyl or aryl. Preferably, the alkynyl alkyl group is of the structure —C≡C—(CH₂)ₙR₅—, wherein n is an integer ranging from 1 to about 10, and R₅ is O, NR₆ or C(O), wherein R₆ is hydrogen, alkyl or aryl. More preferably, the alkynyl alkyl group is of the structure —C≡C—(CH₂)ₙN(H)—, wherein n is an integer ranging from 1 to about 4. Most preferably, the alkynyl alkyl group is of the structure —C≡C—CH₂N(H)—.

“Alkenyl alkyl” refers to a chemical group having the structure —CH═CH—R₄—, wherein R₄ is alkyl or aryl. Preferably, the alkenyl alkyl group is of the structure —CH═CH—(CH₂)ₙR₅—, wherein n is an integer ranging from 1 to about 10, and R₅ is O, NR₆ or C(O), wherein R₆ is hydrogen, alkyl or aryl. More preferably, the alkenyl alkyl group is of the structure —CH═CH—(CH₂)ₙN(H)—, wherein n is an integer ranging from 1 to about 4. Most preferably, the alkenyl alkyl group is of the structure —CH═CH—CH₂N(H)—.

“Functionalized alkyl” refers to a chemical group of the structure —(CH₂)ₙR₇—, wherein n is an integer ranging from 1 to about 10, and R₇ is O, S, NH or C(O). Preferably, the functionalized alkyl group is of the structure —(CH₂)ₙC(O)—, wherein n is an integer ranging from 1 to about 4. More preferably, the functionalized alkyl group is of the structure —CH₂C(O)—.

“Alkoxy” refers to a chemical group of the structure —O(CH₂)ₙR₈—, wherein n is an integer ranging from 2 to about 10, and R₈ is O, S, NH or C(O). Preferably, the alkoxy group is of the structure —O(CH₂)ₙC(O)—, wherein n is an integer ranging from 2 to about 4. More preferably, the alkoxy group is of the structure —OCH₂CH₂C(O)—.

“Thio” refers to a chemical group of the structure —S(CH₂)ₙR₈—, wherein n is an integer ranging from 2 to about 10, and R₈ is O, S, NH or C(O). Preferably, the thio group is of the structure —S(CH₂)ₙC(O)—, wherein n is an integer ranging from 2 to about 4. More preferably, the thio group is of the structure —SCH₂CH₂C(O)—.

“Amino alkyl” refers to a chemical group having an amino group attached to an alkyl group. Preferably an amino alkyl is of the structure —NH(CH₂)ₙNH—, wherein n is an integer ranging from about 2 to about 10. More preferably it is of the structure —NH(CH₂)ₙNH—, wherein n is an integer ranging from about 2 to about 4. Most preferably, the amino alkyl group is of the structure —NH(CH₂)₄NH—.

“Nucleic acid” refers to a polymer comprising 2 or more nucleotides and includes single-, double- and triple stranded polymers. “Nucleotide” refers to both naturally occurring and non-naturally occurring compounds and comprises a heterocyclic base, a sugar, and a linking group, preferably a phosphate ester. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

The phrase “coupled to a support” means bound directly or indirectly thereto including attachment by covalent binding, hydrogen bonding, ionic interaction, hydrophobic interaction, or otherwise.

“Probe” refers to a nucleic acid that can be used to detect, by hybridization, a target nucleic acid. Preferably, the probe is complementary to the target nucleic acid along the entire length of the probe, but hybridization can occur in the presence of one or more base mismatches between probe and target.

“Perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.” In the case of expression monitoring arrays, perfect match probes are typically preselected (designed) to be complementary to particular sequences or subsequences of target nucleic acids (e.g., particular genes). In contrast, in generic difference screening arrays, the particular target sequences are typically unknown. In the latter case, prefect match probes cannot be preselected. The term perfect match probe in this context is to distinguish that probe from a corresponding “mismatch control” that differs from the perfect match in one or more particular preselected nucleotides as described below.

“Mismatch control” or “mismatch probe”, in expression monitoring arrays, refers to probes whose sequence is deliberately selected not to be perfectly complementary to a particular target sequence. For each mismatch (MM) control in a high-density array there preferably exists a corresponding perfect match (PM) probe that is perfectly complementary to the same particular target sequence. In “generic” (e.g., random, arbitrary, haphazard, etc.) arrays, since the target nucleic acid(s) are unknown perfect match and mismatch probes cannot be a priori determined, designed, or selected. In this instance, the probes are preferably provided as pairs where each pair of probes differ in one or more preselected nucleotides. Thus, while it is not known a priori which of the probes in the pair is the perfect match, it is known that when one probe specifically hybridizes to a particular target sequence, the other probe of the pair will act as a mismatch control for that target sequence. It will be appreciated that the perfect match and mismatch probes need not be provided as pairs, but may be provided as larger collections (e.g., 3. 4, 5, or more) of probes that differ from each other in particular preselected nucleotides. While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions. In a particularly preferred embodiment, perfect matches differ from mismatch controls in a single centrally-located nucleotide.

“Labeled moiety” also known as a “detectable moiety” refers to a moiety capable of being detected by the various methods discussed herein or known in the art.

Amido alkyl groups are of the structure —C(O)NR₃R₄—, wherein R₃ is hydrogen, alkyl or aryl, and R₄ is alkyl or aryl. The amido alkyl group is preferably of the structure —C(O)NH(CH₂)ₙR₅—, wherein n is an integer ranging from about 2 to about 10 and R₅ is O, NR₆ or C(O), and wherein R₆ is hydrogen, alkyl or aryl. More preferably, the amido alkyl group is of the structure —C(O)NH(CH₂)ₙN(H)—, wherein n is an integer ranging from about 2 to about 6. Most preferably, the amido alkyl group is of the structure —C(O)NH(CH₂)₄N(H)—.

Alkynyl alkyl groups are of the structure —C≡C—R₄—, wherein R₄ is alkyl or aryl. The alkynyl alkyl group is preferably of the structure —C≡C(CH₂)ₙR₅—, wherein n is an integer ranging from 1 to about 10 and R₅ is O, NR₆ or C(O), and wherein R₆ is hydrogen, alkyl or aryl. More preferably, the alkynyl alkyl group is of the structure —C≡C—(CH₂)ₙN(H)—, wherein n is an integer ranging from 1 to about 4. Most preferably, the alkynyl alkyl group is of the structure —C≡C—CH₂N(H)—.

Alkenyl alkyl groups are of the structure —CH═CH—R₄—, wherein R₄ is alkyl or aryl. The alkenyl alkyl group is preferably of the structure —CH═CH(CH₂)ₙR₅—, wherein n is an integer ranging from 1 to about 10, and R₅ is O, NR₆ or C(O), and wherein R₆ is hydrogen, alkyl or aryl. More preferably, the alkenyl alkyl group is of the structure —CH═CH(CH₂)ₙNH—, wherein n is an integer ranging from 1 to about 4. Most preferably, the alkenyl alkyl group is of the structure —CH═CHCH₂NH—.

Functionalized alkyl groups are of the structure —(CH₂)ₙR₇—, wherein n is an integer ranging from 1 to about 10, and R₇ is O, S, NH, or C(O). The functionalized alkyl group is preferably of the structure —(CH₂)ₙC(O)—, wherein n is an integer ranging from 1 to about 4. More preferably, the functionalized alkyl group is —CH₂C(O)—.

Alkoxy groups are of the structure —O(CH₂)ₙR₈—, wherein n is an integer ranging from 2 to about 10, and R₈ is O, S, NH, or C(O). The alkoxy group is preferably of the structure —O(CH₂)ₙC(O)—, wherein n is an integer ranging from 2 to about 4. More preferably, the alkoxy group is of the structure —OCH₂CH₂C(O)—.

Thio groups are of the structure —S(CH₂)ₙR₈—, wherein n is an integer ranging from 2 to about 10, and R₈ is O, S, NH, or C(O). The thio group is preferably of the structure —S(CH₂)ₙC(O)—, wherein n is an integer ranging from 2 to about 4. More preferably, the thio group is of the structure —SCH₂CH₂C(O)—.

Amino alkyl groups comprise an amino group attached to an alkyl group. Preferably, amino alkyl groups are of the structure —NH(CH₂)ₙNH—, wherein n is an integer ranging from about 2 to about 10. The amino alkyl group is more preferably of the structure —NH(CH₂)ₙNH—, wherein n is an integer ranging from about 2 to about 4. Most preferably, the amino alkyl group is of the structure —NH(CH₂)₂NH—.

A vinyl group refers to an unsaturated ethyl group (—CH═CH—). A carbonyl group is —C(O)—.

The detectable moiety (Q) is a chemical group that provides an signal. The signal is detectable by any suitable means, including spectroscopic, photochemical, biochemical, immunochemical, electrical, optical or chemical means. In certain cases, the signal is detectable by 2 or more means.

The detectable moiety provides the signal either directly or indirectly. A direct signal is produced where the labeling group spontaneously emits a signal, or generates a signal upon the introduction of a suitable stimulus. Radiolabels, such as ³H, ¹²⁵I, ³⁵S, ¹⁴C or ³²P, and magnetic particles, such as Dynabeads™, are nonlimiting examples of groups that directly and spontaneously provide a signal. Labeling groups that directly provide a signal in the presence of a stimulus include the following nonlimiting examples: colloidal gold (40-80 nm diameter), which scatters green light with high efficiency; fluorescent labels, such as fluorescein, texas red, rhodamine, and green fluorescent protein (Molecular Probes, Eugene, Oreg.), which absorb and subsequently emit light; chemiluminescent or bioluminescent labels, such as luminol, lophine, acridine salts and luciferins, which are electronically excited as the result of a chemical or biological reaction and subsequently emit light; spin labels, such as vanadium, copper, iron, manganese and nitroxide free radicals, which are detected by electron spin resonance (ESR) spectroscopy; dyes, such as quinoline dyes, triarylmethane dyes and acridine dyes, which absorb specific wavelengths of light; and colored glass or plastic (e.g., polystyrene, polypropylene, latex, etc.) beads. See U.S. Pat. Nos. 3,817,837; 3,850,752; 3,939,350; 3,996,345; 4,277,437; 4,275,149 and 4,366,241.

A detectable moiety provides an indirect signal where it interacts with a second compound that spontaneously emits a signal, or generates a signal upon the introduction of a suitable stimulus. Biotin, for example, produces a signal by forming a conjugate with streptavidin, which is then detected. See Hybridization With Nucleic Acid Probes. In Laboratory Techniques in Biochemistry and Molecular Biology; Tijssen, P., Ed.; Elsevier: New York, 1993; Vol. 24. An enzyme, such as horseradish peroxidase or alkaline phosphatase, that is attached to an antibody in a label-antibody-antibody as in an ELISA assay, also produces an indirect signal.

A preferred detectable moiety is a fluorescent group. Flourescent groups typically produce a high signal to noise ratio, thereby providing increased resolution and sensitivity in a detection procedure. Preferably, the fluorescent group absorbs light with a wavelength above about 300 nm, more preferably above about 350 nm, and most preferably above about 400 nm. The wavelength of the light emitted by the fluorescent group is preferably above about 310 nm, more preferably above about 360 nm, and most preferably above about 410 nm.

The fluorescent detectable moiety is selected from a variety of structural classes, including the following nonlimiting examples: 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

A number of fluorescent compounds are suitable for incorporation into the present invention. Nonlimiting examples of such compounds include the following: dansyl chloride; fluoresceins, such as 3,6-dihydroxy-9-phenylxanthhydrol; rhodamineisothiocyanate; N-phenyl-1-amino-8-sulfonatonaphthalene; N-phenyl-2-amino-6-sulfonatonaphthanlene; 4-acetamido-4-isothiocyanatostilbene-2,2′-disulfonic acid; pyrene-3-sulfonic acid; 2-toluidinonapththalene-6-sulfonate; N-phenyl, N-methyl2-aminonaphthalene-6-sulfonate; ethidium bromide; stebrine; auromine-0,2-(9′-anthroyl)palmitate; dansyl phosphatidylethanolamin; N,N′-dioctadecyl oxacarbocycanine; N,N′-dihexyl oxacarbocyanine; merocyanine, 4-(3′-pyrenyl)butryate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis[2-(4-methyl-5-phenyl oxazolyl)]benzene; 6-dimethylamino-1,2-benzophenzin; retinol; bis(3′-aminopyridium)-1,10-decandiyl diiodide; sulfonaphthylhydrazone of hellibrienin; chlorotetracycline; N-(7-dimethylamino-4-methyl-2-oxo-3-chromenyl)maleimide; N-[p-(2-benzimidazolyl)phenyl]maleimide; N-(4-fluoranthyl)maleimide; bis(homovanillic acid); resazarin; 4-chloro-7-nitro-2,1,3-benzooxadizole; merocyanine 540; resorufin; rose bengal and 2,4-diphenyl-3(2H)-furanone. Preferably, the fluorescent detectable moiety is a fluorescein or rhodamine dye.

Another preferred detectable moiety is colloidal gold. The colloidal gold particle is typically 40 to 80 nm in diameter. The colloidal gold may be attached to a labeling compound in a variety of ways. In one embodiment, the linker moiety of the nucleic acid labeling compound terminates in a thiol group (—SH), and the thiol group is directly bound to colloidal gold through a dative bond. See Mirkin et al. Nature 1996, 382, 607-609. In another embodiment, it is attached indirectly, for instance through the interaction between colloidal gold conjugates of antibiotin and a biotinylated labeling compound. The detection of the gold labeled compound may be enhanced through the use of a silver enhancement method. See Danscher et al. J. Histotech 1993, 16, 201-207.

Detectable moieties may be incorporated into a nucleotide analog such that the label is directly incorporated into a nucleic acid as the nucleotide analog is incorporated into the nucleic acid. Alternatively, a detectable moiety may be incorporated into a nucleic acid by first incorporating nucleotide analogs with connecting groups desinged to allow coupling of the detectable moiety to the connecting group. Such labeling may be desireable in cases where certain nucleotide-label conjugates might behave as poor enzyme substrates. Such is not unexpected with large hydrophobic fluor groups, many of which are discussed here in accordance with the present invention.

An example of what is sometimes termed “post-amplification” labeling is shown in, for example, Trevisiol, et al., Eur. J. Org. Chem. 2000, 211-217 and Trevisiol et al., Nucleosides and Nucleotides, 18 (4 & 5) 979-980 (1999) (collectively “Trevisiol”), each of which is incorporated herein by reference. In Trevisiol, the following ribonucleoside derivative is disclosed: 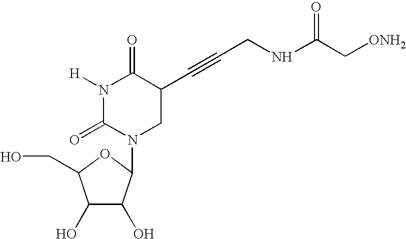
Trevisiol discloses that the triphosphate derivative of the above amino-oxy ribonucleoside can act as a substrate of T7 RNA polymerase and be incorporated with that enzyme into RNA. After incorporation into RNA, the amino-oxy functionality can be reacted with the following aldehyde functionalized fluorophore to provide labeled RNA:

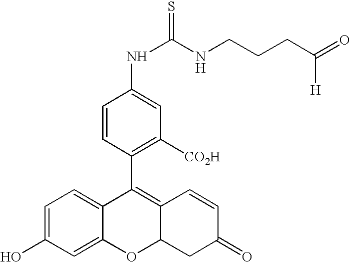

cDNA labelling by direct incorporation of Cy3- or Cy5-modified nucleotides has been compared with labelling via incorporation of 5-(3-aminoallyl)-dUTP (aa-dUTP) and subsequent coupling of the aa-modified cDNA to Cy3 or Cy5 (or other) dyes provided with N-hydroxysuccinimide ester moieties (References 1-5). This has been done in RNA labeling as well (6, 7), using IVT, where improvements in average length and yield of transcript may be achieved relative to labeled NTPs. In the case of Cye dyes, aminoallyl labeling can result in higher labeling efficiency and consistency, and reduced cost.”

### References

1. Forghani, B., Yu, G. J. and Hurst, J. W. (1991) Comparison of biotinylated DNA and RNA probes for rapid detection of varicella-zoster virus genome by in situ hybridization. J. Clin. Microbiol., 29, 583-591. —use commerc. AA-UTP for IVT lab'ng.

2. Nimmakayalu, M., Henegariu, O., Ward, D. C. and Bray-Ward, P. (2000) Simple method for preparation of fluor/hapten-labeled dUTP. Biotechniques, 28, 518-522.

3. Richter, A., Schwager, C., Hentze, S., Ansorge, W., Hentze, M. W., and Muckenthaler, M. (2002) Comparison of fluorescent tag DNA labeling methods used for expression analysis by DNA microarrays. Biotechniques, 33, 620-628, 630.

4. Yu, J., Othman, M. I., Farjo, R., Zareparsi, S., MacNee, S. P., Yoshida, S. and Swaroop, A. (2002) Evaluation and optimization of procedures for target labeling and hybridization of cDNA microarrays. Mol. Vis., 8, 130-137.

5. Xiang, C. C., Kozhich, O. A., Chen, M., Inman, J. M., Phan, Q. N., Chen, Y. and Brownstein, M. J. (2002) Amine-modified random primers to label probes for DNA microarrays. Nat. Biotechnol. 20, 738-742.

6. Luehrson, K. R.; Baum, M. P. Biotechniques 1987, 5, 660-2.

7. 't Hoen, P. A. C.; de Kort, F.; van Ommen, G. J. B.; den Dunnen, J. T. (2003) Fluorescent labeling of cRNA for microarray applications. Nucleic Acids Research 31, e20. (IVT cRNA labeling).

In accordance with one aspect of the present invention, a method is presented for post-incorporation labeling of cRNA having the steps of providing a cDNA template having a T7 RNA promoter. In preferred embodiments of the present invention, the template corresponds to a mRNA whose transcription level is to be determined by hybridization of the labeled cRNA to a nucleic acid array.

The template is transcribed with a mixture of nucleotides, the mixture having a nucleotide analog which is selected from the group consisting of 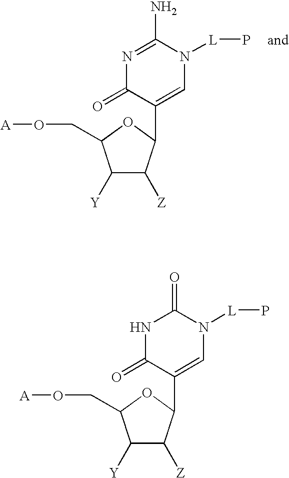
wherein A is H or a functional group that permits the attachment of the nucleotide analog to a nucleic acid; Y and Z are independently H or OH; L is linker group; and P is a connecting group to provide primed cRNA.

As disclosed in accordance with one aspect of the present invention, the linker group L is selected to provide a linking function by which appropriate spacing of the detectable group Q group from the base group is provided at such a length and in such a configuration as to allow an appropriate assay to be performed on the Q group.

The linker moiety (L) of the nucleic acid labeling compound is covalently bound to the heterocycle (H_{c}) at one terminal position. The linker group, in accordance with the present invention, has a structure that is sterically and electronically suitable for incorporation into a nucleic acid. Nonlimiting examples of linker moieties include amido alkyl groups, alkynyl alkyl groups, alkenyl alkyl groups, functionalized alkyl groups, alkoxyl groups, thio groups, vinyl groups, alkoxy groups, amino alkyl groups and combinations of the above.

P is a connecting group, in accordance with the present invention. P is a chemical group or moiety selected to allow detectable moiety to be specifically coupled to the cRNA after the nucleotide analog containing P is incorporated into cRNA or any nucleic acid.

Following transcription of the primed cRNA, the primed cRNA is reacted with a detectable group reagent, wherein said detetable group reagent comprises a chemical moiety which is capable of specifically reacting with said P group to allow coupling of the detectable group to said primed cRNA. The detectable group reagent is prepared in accordance with the present invention by derivatizing a detectable moiety as described and defined here so that the derivatized detectable moiety will specifically react with the P group of the nucleotide analog. For example, in accordance with the present invention, if the P group is a nucleophile, the detectable moiety reagent comprises an electrophile or electrophilic group, allowing coupling of the detectable moiety to the incorporated nucleotide analog. Correspondingly, if the detectable moiety reagent comprises a nucleophile or nucleophilic group, the P group is electrophilic, again allowing specific coupling.

According to the present invention, both the P group and the detectable moiety reagent must be stable and relatively non-reactive to other components and reagents used in the methods disclosed herein under the conditions, e.g., temperature and pH, employed. Moreover, the P group and detectable moiety reagent must be reactive towards one another under relatively mild conditions.

In preferred embodiments of the present invention, the P group comprises a terminal moiety selected from the group consisting of —NH₂, —SH, —ONH₂, —CO₂H, —C(O)R, wherein R is an alkyl, aryl or functionalized alkyl group and —C(R)HX, wherein X is a halogen.

In still other preferred embodiments of the present invention, the chemical moiety of the detetable group reagent is selected from the group consisting of —NH₂, —SH, ONH₂, —CO₂H, C(O)R, wherein R is H, alkyl, aryl or a functionalized alkyl group, and —C(R)HX, wherein X is a halogen.

In a particularly preferred embodiment of the present invention, P comprises —NH₂ and the detectable group comprises —C(R)HX. X is preferably chloro, bromo or iodo.

In particularly preferred embodiments of the present invention, -L-P is

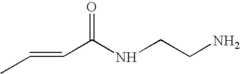

Accordingly, a particularly preferred nucleotide analog of the present invention is selected from the group consisting of

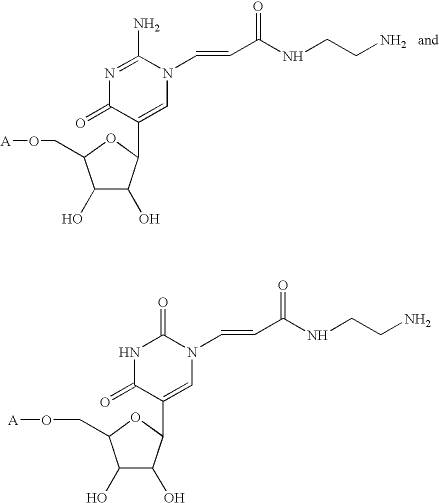

In preferred embodiments of the present invention, A is a triphosphate group with counterions. The counterions are selected from the group consisting of H⁺, Na⁺, Li⁺, K⁺, and NH₄⁺.

Also presented in accordance with the present invention, are compounds and reagents comprising nucleotide analogues: 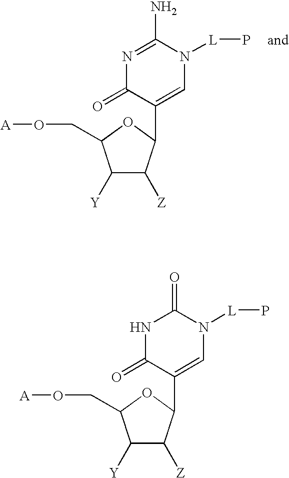
wherein A is H or a functional group that permits the attachment of the nucleotide analog to a nucleic acid; Y and Z are independently H or OH; L is linker group; and P is a connecting group.

In the aboved nucleotide analogs it is preferred that -L-P is

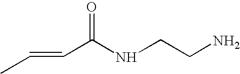

It is preferred that A is a triphosphate group with counterions and that the counterions selected from the group consisting of H⁺, Na⁺, Li⁺, K⁺, and NH₄⁺.

Two preferred nucleotide analogs according to the present invention are:

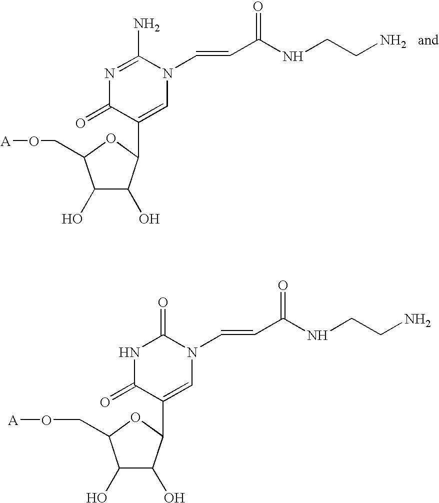

The present invention also contemplates production of a nucleic acid derivative produced by coupling a nucleotide analog with a nucleic acid. Labeled nucleic acid is produced by reacting the incorporated nucleic acid analog with a detectable moiety reagent. According to the claimed invention, a hybridization product is formed comprising the labeled nucleic acid above bound to a complementary probe.

It is preferred that in the hybridization product above the probe is bound to a solid support. Preferably the solid support is glass.

It is important to be noted, for purposes of the present invention, that the compounds of the present invention are not limited to use in post incorporation labeling of cRNA. In accordance with another aspect of the present invention, general-purpose labeling of nucleic acids, including DNA sequences, using nucleotide analogs, which are disclosed in accordance with the present invention, can be carried out by a variety of methods, including via nick translation (Leary, J. J.; Brigati, D. J.; Ward, D. C. Proc. Nat. Acad. Sci. USA (1983) 80, 4045-4049), random priming (Klevan, L.; Gebeyehu, G. Methods Enzymol. 1990, 184, 561), PCR amplification (Dennis, L. Y.-M.; Mehal, W. Z.; Fleming, K. A. In: PCR Protocols: A Guide to Methods and Applications (1990) and Innis, M. A.; Gelfand, D. H.; Sninsky, J. J.; White, T. J.; Editors. Academic Press, San Diego, Calif. (1990). pp. 113-8), reverse transcription (Schena, M.; Shalon, D.; Davis, R. W.; Brown, P. O. Science 1995, 270, 467-470) or 3′-end labeling with terminal transferase (Figeys, D.; Renborg, A.; Dovichi, N. J. Anal. Chem. 1994, 66, 4382-3 and Wang, D. G.; et al. Science 1998, 280, 1077-1082), RNA molecules can be labeled via in vitro transcription (McCracken, S. Focus 1986, 7, 5-8 and Lockhart, D. J.; Dong., H.; Byrne, M. C.; Follettie, M. T.; Gallo, M. V.; Chee, M. S.; Mittmann, M.; Wang, C.; Kobayashi, M.; Horton, H.; Brown, E. L. Nature Biotechnol. 1996, 14, 1675-1680, or 3′-labeling with terminal transferase (Rosenmyer, V.; Laubrock, A.; Seibl, R. Anal. Biochem. 1995, 224, 446-449) and polyA polymerase (Martin, G.; Keller, W. RNA 1998, 4, 226-30). Numerous practical guides are available, describing typical labeling protocols for DNA probes. Mundy, C. R.; Cunningham, M. W.; Read, C. A. In: Essential Molecular Biology (2nd Edition) Brown, T. A. (Ed.) (2001), Oxford University Press, Oxford, UK. p 63-107; Rapley, R., Ed., The Nucleic Acid Protocols Handbook, Humana Press, Totowa, N.J., 2000. p117-173, Kessler, C. In: Nonisotopic Probing, Blotting and Sequencing, Kricka, L. J., Ed., Academic Press, 1995, 41-109 and Keller, G. H. In: DNA Probes, 2^{nd} Ed. , Keller, G. H., Manak, M. M., Eds., Stockton Press, N.Y., 1993, p 173-p198. Also, various array based applications are disclosed. Schena, M. (Ed.) DNA Microarrays. A Practical Approach. Oxford University Press, UK, 1999 and Rampal, J. B. (Ed.) DNA Arrays. Humana Press, Totowa, N.J., 2001. Each of these references is incorporated herein by reference. In accordance with one aspect of the present invention, the nucleotide analogs, incorporated as disclosed above, may be labeled with a detectable moiety reagent as disclosed herein.

The present invention also provides method for post-incorporation labeling of nucleic acids into which nucleotide analogs of the present invention are incorporated, the method provides: providing a nucleic acid; incorporating a nucleotide analog into said nucleic acid, said nucleotide analog selected from the group consisting of 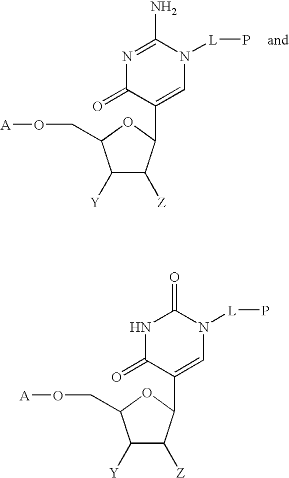
wherein A is H or a functional group that permits the attachment of the nucleotide analog to a nucleic acid; Y and Z are independently H or OH; L is linker group; and P is a connecting group to provide primed nucleic acid; and reacting said primed nucleic acid with a detectable group reagent, wherein said detetable group comprises a chemical moiety which is capable of specifically reacting with said P group to allow coupling of the detectable group to said primed nucleic acid.

It is preferred that the P group comprises a terminal moiety seleceted from the group consisting of —NH₂, —SH, —ONH₂, —CO₂H, —C(O)R, wherein R is an alkyl, aryl or functionalized alkyl group and —C(R)HX, wherein X is a halogen. It is also preferred that the detetable group reagent comprises a moiety selected from the group consisting of —NH₂, —SH, ONH₂, —CO₂H, C(O)R, wherein R is H, alkyl, aryl or a functionalized alkyl group, and —C(R)HX, wherein X is a halogen.

In a particular preferred embodiment, P comprises —NH₂ and the detectable group comprises —C(R)HX.

In a preferred embodiment of the method, -L-P is

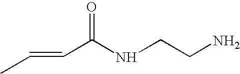

In a preferred embodiment of the present method, the nucleotide analog is selected from the group consisting of

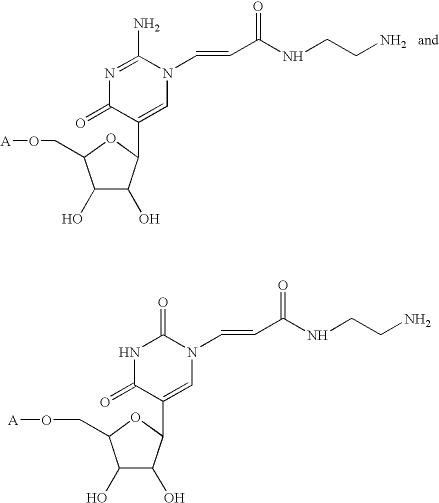

In a preferred embodiment of the present method, A is a triphosphate group with counterions. The counterions are preferably selected from the group consisting of H⁺, Na⁺, Li⁺, K⁺, and NH₄⁺.

In a particularly preferred embodiment of the method the nucleic acid is double stranded DNA and said step of incorporating is performed with the enzyme terminal transferase. In this preferred embodiment, A is a triphosphate group with appropriate counterions, Y is OH and Z is H.

The present invention also contemplates other compounds and methods which may be employed for post-incorporation labeling of a nucleic acid. Other means contemplated by the present invention include enzymatic attachment of detectable moieties to nucleotide derivatives incorporated into a nucleic acid, for example a cRNA.

All patents, patent applications, and literature cited in the specification are hereby incorporated by reference in their entirety. In the case of any inconsistencies, the present disclosure, including any definitions therein will prevail.

The invention has been described with reference to various specific and preferred embodiments and techniques. However, it should be understood that many variations and modifications may be made while remaining within the spirit and scope of the invention.

1. A method for post-incorporation labeling of cRNA, said method comprising the steps of
providing a cDNA template having a T7 RNA promoter,
transcribing said template with a mixture of nucleotides, said mixture comprising a nucleotide analog, said analog selected from the group consisting of 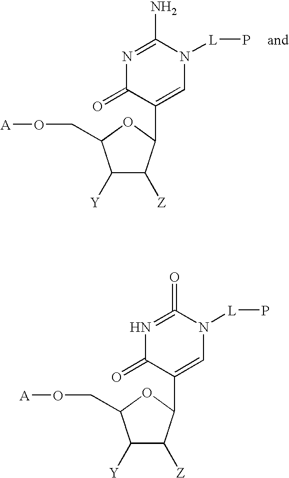
wherein A is triphosphate or α-thio triphosphate that permits the attachment of the nucleotide analog to a nucleic acid; Y and Z are independently H or OH; L is linker group; and P is a connecting group to provide primed cRNA; and
reacting said primed cRNA with a detectable group reagent, wherein said detetable group reagent comprises a chemical moiety which is capable of specifically reacting with said P group to allow coupling of the detectable group to said primed cRNA.

2. A method according to claim 1 wherein said P group comprises a terminal moiety seleceted from the group consisting of —NH₂, —SH, —ONH₂, —CO₂H, —C(O)R, wherein R is H, alkyl, aryl or functionalized alkyl group and —C(R)HX, wherein X is a halogen.

3. A method according to claim 1 wherein said chemical moiety of said detetable group comprises a moiety selected from the group consisting of —NH₂, —SH, ONH₂, —CO₂H, C(O)R, wherein R is H, alkyl, aryl or a functionalized alkyl group, and —C(R)HX, wherein X is a halogen.

4. A method according to claim 1 wherein P comprises —NH₂ and said detectable group comprises —C(R)HX.

5. A method according to claim 1 wherein -L-P is

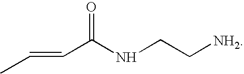

6. A method according to claim 1 wherein said nucleotide analog is selected from the group consisting of

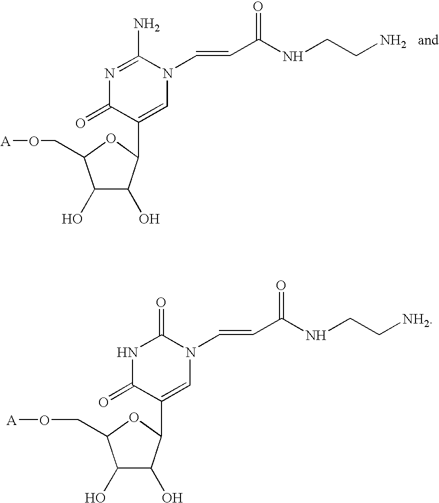

7. A method according to claim 6 wherein A is a triphosphate group with counterions, said counterions selected from the group consisting of H⁺, Na⁺, Li⁺, K⁺, and NH₄⁺.

8. A nucleotide analog having the following formula: 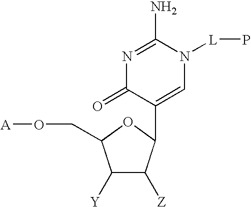
wherein A is H or a functional group that permits the attachment of the nucleotide analog to a nucleic acid; Y and Z are independently H or OH; L is linker group; and P is a connecting group.

9. A nucleotide analog according to claim 8 wherein -L-P is

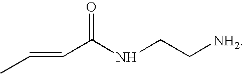

10. A nucleotide analog according to claim 8 wherein A is a triphosphate group with counterions, said counterions selected from the group consisting of H⁺, Na⁺, Li⁺, K⁺, and NH₄⁺.

11. A nucleotide analog according to claim 8 having the formula

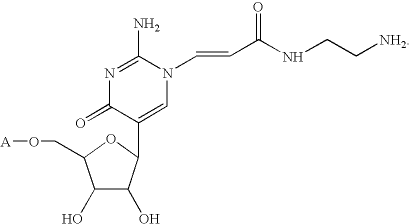

12. A nucleic acid derivative produced by coupling a nucleotide analog according to claim 8 with a nucleic acid.

13. A labeled nucleic acid produced by reacting the nucleic acid derivative of claim 12 with a detectable moiety reagent.

14. A hybridization product comprising the labeled nucleic acid according to claim 13 bound to a complementary probe.

15. A hybridization product according to claim 14 wherein the probe is bound to a solid support.

16. A hybridization product according to claim 15 wherein said solid support is glass.

17. A nucleotide analog having the following formula: 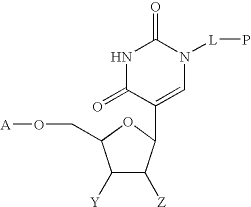
wherein A is H or a functional group that permits the attachment of the nucleotide analog to a nucleic acid; Y and Z are independently H or OH; L is linker group; and P is a connecting group.

18. A nucleotide analog according to claim 17 wherein -L-P is

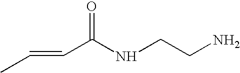

19. A nucleotide analog according to claim 17 wherein A is a triphosphate group with counterions, said counterions selected from the group consisting of H⁺, Na⁺, Li⁺, K⁺, and NH₄⁺.

20. A nucleotide analog according to claim 17 having the formula

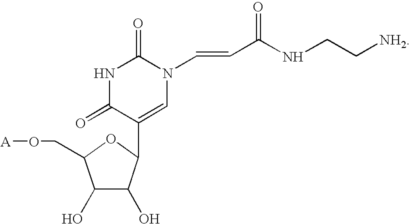

21. A nucleic acid derivative produced by coupling a nucleotide analog according to claim 17 with a nucleic acid.

22. A labeled nucleic acid produced by reacting the nucleic acid derivative of claim 21 with a detectable moiety reagent.

23. A hybridization product comprising the labeled nucleic acid according to claim 22 bound to a complementary probe.

24. A hybridization product according to claim 23 wherein the probe is bound to a solid support.

25. A hybridization product according to claim 24 wherein said solid support is glass.

26. A method of post-incorporation labeling of a nucleic acid comprising the steps of
providing a nucleic acid;
incorporating a nucleotide analog into said nucleic acid, said nucleotide analog selected from the group consisting of 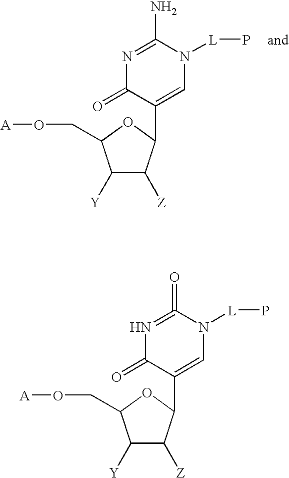
wherein A is H or a functional group that permits the attachment of the nucleotide analog to a nucleic acid; Y and Z are independently H or OH; L is linker group; and P is a connecting group to provide primed nucleic acid; and
reacting said primed nucleic acid with a detectable group reagent, wherein said detetable group comprises a chemical moiety which is capable of specifically reacting with said P group to allow coupling of the detectable group to said primed nucleic acid.

27. A method according to claim 26 wherein said P group comprises a terminal moiety seleceted from the group consisting of —NH₂, —SH, —ONH₂, —CO₂H, —C(O)R, wherein R is H, alkyl, aryl or functionalized alkyl group and —C(R)HX, wherein X is a halogen.

28. A method according to claim 26 wherein said chemical moiety of said detetable group comprises a moiety selected from the group consisting of —NH₂, —SH, ONH₂, —CO₂H, C(O)R, wherein R is H, alkyl, aryl or a functionalized alkyl group, and —C(R)HX, wherein X is a halogen.

29. A method according to claim 26 wherein P comprises —NH₂ and said detectable group comprises —C(R)HX.

30. A method according to claim 26 wherein -L-P is

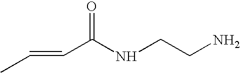

31. A method according to claim 26 wherein said nucleotide analog is selected from the group consisting of

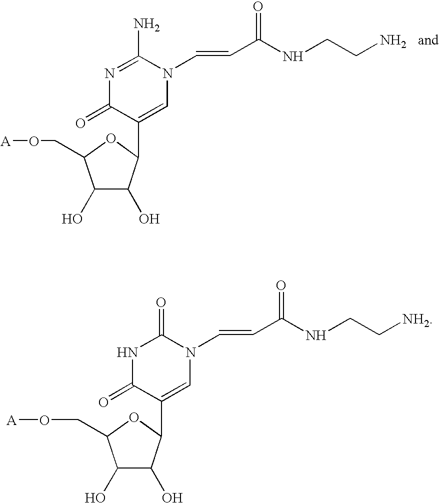

32. A method according to claim 26 wherein A is a triphosphate group with counterions, said counterions selected from the group consisting of H⁺, Na⁺, Li⁺, K⁺, and NH₄⁺.

33. A method according to claim 26 wherein said nucleic acid is double stranded DNA and said step of incorporating is performed with the enzyme terminal transferase.

34. A method according to claim 33 wherein A is a triphosphate group with appropriate counterions, Y is OH and Z is H.

35. A method according to claim 1 wherein said cDNA template corresponds to a mRNA whose level of expression is to be tested by hybridization of the labeled cRNA to a nucleic acid array.
